(19) United States
(12) Patent Application Publication
(10) Pub. No.: US 2021/0145830 A1
(43) Pub. Date: May 20, 2021

(54) MATERIALS AND METHODS FOR TREATING CANCER

(71) Applicants: Mayo Foundation for Medical Education and Research, Rochester, MN (US); Sumitomo Dainippon Pharma Oncology, Inc., Cambridge, MA (US)

(72) Inventors: Saad J. Kenderian, Rochester, MN (US); Neil E. Kay, St. Louis Park, MN (US); Reona Sakemura, Rochester, MN (US); Steven L. Warner, Sandy, UT (US); David J. Bearss, Alpine, UT (US); Jason Marc Foulks, Sandy, UT (US); Lars Mouritsen, South Jordan, UT (US); Clifford J. Whatcott, West Jordan, UT (US)

(21) Appl. No.: 17/045,411

(22) PCT Filed: Apr. 5, 2019
(86) PCT No.: PCT/US2019/026108

§ 371 Date: Oct. 5, 2020

Related U.S. Application Data
(60) Provisional application No. 62/753,712, filed on Oct. 31, 2018.
(60) Provisional application No. 62/653,376, filed on Apr. 5, 2018.

(51) Int. Cl. A61K 31/506; A61P 35/00; C12N 5/0783

(52) U.S. Cl. CPC A61K 31/506; C12N 5/0636; A61P 35/00

(57) ABSTRACT

This document provides methods for determining biological age of mammalian subjects or assessing whether the subjects are aging normally. Also provided herein are methods for determining whether therapeutic treatment or other interventions for reversing or slowing down aging are effective. This document also provides methods and materials for slowing the progression of biological aging.

### CROSS-REFERENCE TO RELATED APPLICATIONS

This application claims priority to U.S. Application Ser. No. 62/753,712, filed on Oct. 31, 2018 and U.S. Application Ser. No. 62/653,376, filed on Apr. 5, 2018. The disclosures of the prior applications are considered part of the disclosure of this application, and are incorporated in their entirety into this application.

### BACKGROUND

### 1. Technical Field

This document relates to methods and materials involved in treating cancer. For example, this document provides methods and materials for using one or more inhibitors of a TAM (Tyro3, Axl, and Mer) receptor tyrosine kinase (RTK) in combination with an adoptive cell therapy (e.g., a chimeric antigen receptor T cell therapy) to alter one or more functions of a T cell and/or to enhance T cell expansion to treat cancer in a mammal (e.g., a human).

### 2. Background Information

Chimeric antigen receptor (CAR) T cell therapy is an effective modality for the treatment of cancer. CD19 directed CAR T cells (CART19) have recently emerged as a potent and potentially curative therapy in relapsed refractory acute lymphoblastic leukemia (ALL). However, the complete response (CR) rate in diffuse large B cell lymphoma (DLBCL) or chronic lymphocytic leukemia (CLL) after CART19 therapy is much lower at approximately 30%. Furthermore, a limiting toxicity after CART cell therapy is the development of cytokine release syndrome, and mortality has been reported with this syndrome.

### SUMMARY

This document provides methods and materials involved in treating cancer. For example, one or more TAM RTK inhibitors (e.g., one or more AXL inhibitors) can be administered to a mammal (e.g., a human) having cancer in combination with an adoptive cell therapy (e.g., a CART cell therapy) to alter one or more functions of a T cell and/or to enhance T cell expansion to treat cancer in a mammal (e.g., a human). For example, one or more TAM RTK inhibitors can be used in combination with a CART cell therapy to alter (e.g., enhance or suppress) one or more functions of a CART cell and/or to enhance CART cell expansion.

As demonstrated herein, inhibition of AXL in combination with CART cell therapy altered (e.g., enhanced or suppressed) CART cell functions and enhanced T cell expansion. For example, inhibition of AXL in combination with CART cell therapy promoted (e.g., enhanced) T helper (Th) cell differentiation, decreased (e.g., suppressed) expression of inhibitory receptors by CART cells, decreased regulatory T cells, and decreased (e.g., suppressed) release of CRS critical cytokines by CART cells. Also as demonstrated herein, inhibition of AXL in combination with CART cell therapy prevented CART cell exhaustion. Thus, AXL inhibitors can be incorporated into adoptive T cell therapies (e.g., CART cell therapies) to treat, for example, cancer.

In general, one aspect of this document features a method for treating cancer. The method comprises (or consists essentially of, or consisting of) administering an AXL inhibitor to a mammal having cancer that received an adoptive T cell therapy to treat the cancer. The mammal can be a human. The cancer can be a lymphoma. The lymphoma can be a diffuse large B cell lymphoma. The cancer can be a leukemia. The leukemia can be an acute lymphoblastic leukemia or a chronic lymphocytic leukemia. The AXL inhibitor can be TP0903 (e.g., freebase or a pharmaceutically acceptable salt thereof). The TP0903 can be administered to the mammal at a daily dose of about 1-37 mg/m². The TP0903 can be administered to the mammal at a daily dose of about 1-25 mg/m². The TP0903 can be administered to the mammal at a daily dose of about 1-75 mg. The TP0903 can be administered to the mammal at a daily dose of about 1-50 mg. T cells of the adoptive T cell therapy can comprise a chimeric antigen receptor. The chimeric antigen receptor can target a tumor-associated antigen. The tumor-associated antigen can be CD19. In some cases, more Th2 cells of the mammal can differentiate into Th1 cells than in a comparable mammal that received the adoptive T cell therapy in the absence of the administration of the AXL inhibitor. T cells of the adoptive T cell therapy can express less inhibitory receptors than T cells of an adoptive T cell therapy in a comparable mammal that did not receive the administration of the AXL inhibitor. The inhibitory receptors can be selected from the group consisting of programmed cell death protein 1 (PD1) receptors, T cell immunoglobulin and mucin-domain containing 3 (TIM3) receptors, and lymphocyte activation gene 3 (LAG3) receptors. The adoptive T cell therapy can cause less expression of cytokines associated with cytokine release syndrome within the mammal than the level of expression of the cytokines within a comparable mammal that received the adoptive T cell therapy in the absence of the administration of the AXL inhibitor. The cytokines can be selected from the group consisting of MCP1, IL-6, IL-10, and MIP-b. A greater level of T cell expansion can occur within the mammal than the level of T cell expansion that occurs within a comparable mammal that received the adoptive T cell therapy in the absence of the administration of the AXL inhibitor.

In another aspect, this document features a method for treating cancer. The method comprises (or consists essentially of, or consisting of) administering T cells to a mammal having cancer, wherein the T cells were contacted with an AXL inhibitor. The mammal can be a human. The cancer can be a lymphoma. The lymphoma can be a diffuse large B cell lymphoma. The cancer can be a leukemia. The leukemia can be an acute lymphoblastic leukemia or a chronic lymphocytic leukemia. The AXL inhibitor can be TP0903. The TP0903 can be administered to the mammal at a daily dose of about 1-37 mg/m². The TP0903 can be administered to the mammal at a daily dose of about 1-25 mg/m². The TP0903 can be administered to the mammal at a daily dose of about 1-75 mg. The TP0903 can be administered to the mammal at a daily dose of about 1-50 mg. The T cells can comprise a chimeric antigen receptor. The chimeric antigen receptor can target a tumor-associated antigen. The tumor-associated antigen can be CD19. In some cases, more Th2 cells of the mammal can differentiate into Th1 cells than in a comparable mammal that received T cells not contacted with the AXL inhibitor. The T cells can express less inhibitory receptors than comparable T cells not contacted with the AXL inhibitor. The inhibitory receptors can be selected from the group consisting of programmed cell death protein 1 (PD1) receptors, T cell immunoglobulin and mucin-domain containing 3 (TIM3) receptors, and lymphocyte activation gene 3 (LAG3) receptors. Administration of the T cells can cause less expression of cytokines associated with cytokine release syndrome within the mammal than the level of expression of the cytokines within a comparable mammal that received the T cells not contacted with the AXL inhibitor. The cytokines can be selected from the group consisting of MCP1, IL-6, IL-10, and MIP-b. A greater level of T cell expansion can occur within the mammal than the level of T cell expansion that occurs within a comparable mammal that received T cells not contacted with the AXL inhibitor.

In another aspect, this document features an ex vivo method for expanding T cells for treating cancer. The method comprises (or consists essentially of, or consisting of) contacting T cells with an AXL inhibitor. The T cells can be human T cells. The cancer can be a lymphoma. The lymphoma can be a diffuse large B cell lymphoma. The cancer can be a leukemia. The leukemia can be an acute lymphoblastic leukemia or a chronic lymphocytic leukemia. The AXL inhibitor can be TP0903. The T cells can comprise a chimeric antigen receptor. The chimeric antigen receptor can target a tumor-associated antigen. The tumor-associated antigen can be CD19. Th2 cells of the T cells can differentiate into Th1 cells. The T cells can express less inhibitory receptors than comparable T cells not contacted with the AXL inhibitor. The inhibitory receptors can be selected from the group consisting of programmed cell death protein 1 (PD1) receptors, T cell immunoglobulin and mucin-domain containing 3 (TIM3) receptors, and lymphocyte activation gene 3 (LAG3) receptors. Administration of the T cells to a mammal can cause less expression of cytokines associated with cytokine release syndrome within the mammal than the level of expression of the cytokines within a comparable mammal that received the T cells not contacted with the AXL inhibitor. The cytokines can be selected from the group consisting of MCP1, IL-6, IL-10, and MIP-b. A greater level of T cell expansion can occur than the level of T cell expansion that occurs with comparable T cells not contacted with the AXL inhibitor.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains a graph showing CART19 cytotoxicity in the presence TP0903. CART19 or untransduced T cells (UTDs) were co-cultured at different effector-to-target ratios (E:T) with luciferase-positive Jeko with increasing doses of TP0903 (TP). At 24 hours, cell killing was assessed by luminescence. The receiving combination therapy with CART19 and Axl inhibition with TP0903 showed robust killing at all E:T ratios.

FIG. 2 contains an exemplary timeline for a CART19 proliferation assay in the presence of TP0903. UTDs or CART19 were incubated with Jeko in the presence of TP0903 in 1:1 ratio. At day 5, total T cell numbers were evaluated with CountBright quantification.

FIG. 3 contains a graph showing that TP0903 does not inhibit CART specific proliferation.

FIG. 4 contains graphs showing that TP0903 does not inhibit CART cell specific proliferation.

FIG. 5 contains graphs showing the number of CD4 and CD8 cells positively stained for cluster of differentiation 107a (CD107a) or granulocyte-macrophage colony-stimulating factor (GM-CSF) in degranulation assays. Axl inhibition with TP0903 did not inhibit T cell specific degranulation or intracellular cytokines.

FIG. 6 contains graphs showing the number of CD4 cells positively stained for interleukin-2 (IL-2) or interleukin-4 (IL-4) in degranulation assays.

FIG. 7 contains an exemplary timeline for a cytokine assay in the presence of TP0903. UTDs or CART19 were incubated with Jeko in the presence of TP0903 in the 1 to 1 ratio. At day 3, supernatant was harvested and analyzed by 32-plex Luminex array.

FIG. 8 contains graphs showing that TP0903 maintains CART Th1 specific cytokines interferon gamma (IFN-γ) and tumor necrosis factor alpha (TNF-α).

FIG. 9 contains graphs showing that TP0903 suppresses CART Th2 specific cytokines interleukin 4 (IL-4) and interleukin 5 (IL-5).

FIG. 10 contains graphs showing that TP0903 suppresses CART—cytokine release syndrome (CRS) of key cytokines such as monocyte chemoattractant protein 1 (MCP1), interleukin 6 (IL-6), interleukin 10 (IL-10), and macrophage inflammatory protein b (MIP-b).

FIG. 11 contains an exemplary timeline for an in vitro CART19 exhaustion assay in the presence of TP0903. T cells were incubated with target cells at a 1:5 ratio (experiments were done using JEKO cells as well as primary B cells). The low E:T ratio was used to induce a state of dysfunction of CART cells. Cells were cultured in the presence of increasing doses of TP0903. On day 5 of co-culture, cells were harvested, and expression of inhibitory receptors was analyzed. Co-culture in the presence of TP0903 reduces expression of PD1, TIM3, and LAG3 inhibitory receptors.

FIG. 12 contains graphs showing that TP0903 reduces exhaustion receptors on CART cells.

FIG. 13 contains graphs showing T cell expansion of CART19 cells or UTDs with DMSO (control figure).

FIG. 14 contains graphs showing CART19 or untransduced T cell expansion with or without varied concentrations of TP0903. Low dose TP0903 enhanced the expansion of CART19 and UTDs during ex vivo manufacture of CARTs. These results demonstrate that TP0903 can be used during the process of CART manufacturing.

FIG. 15 contains a western blot of signaling molecules in stimulated T cells and unstimulated T cells with or without TP0903. Jurkat cells were used as a control.

FIGS. 16A-B. A head-to-head comparison of two different combination therapy. UTDs (A) or CART19 (B) were co-cultured at different effector-to-target ratios (E:T) with luciferase-positive CD19⁺ cell line in the presence of DMSO, 30 nM TP0903, or 100 nM ibrutinib. Cell killing was assessed by luminescence at 24 hours. The combination of axl inhibition with TP0903 and CART9 resulted in more synergistic killing compared to the combination of ibrutinib and CART19.

FIG. 17. Increasing doses of TP0903 reduced regulatory CART cells when CART19 are co-cultured with their targets.

FIG. 18. TP0903 resulted in an increase of phospho LCK in stimulated CART cells that is dose dependent, reduction in T-bet, reduction in GATA-3, reduction in P38, and reduction in P-JNK.

FIGS. 19A-F. A. Inhibitory receptors expression in naïve T cells after TCR activation with CD3 stimulation in the increasing dose of TP0903. Naïve T cells were incubated with anti CD3/CD28 beads at a 1 to 3 ratio in the presence of TP0903. At day 3, T cells were stained with PD-1, TIM-3, LAG-3, and ICOS. B. TP0903 specifically reduced Treg. Teff cells were isolated by negative selection using Human T Cell Isolation Kit. Tregs were isolated by negative selection using the human CD4⁺CD127^{low}CD25⁺ Treg isolation kit. Teff and Treg were stimulated with anti CD3/CD28 beads and 400 U/ml of IL-2. At day 5, total T cells were evaluated via CountBright quantification. C. CART19 cytokine assay in the presence TP0903. UTDs or CART19 were incubated with Jeko in the presence of TP-0903 at a 1 to 1 ratio. Supernatant was harvested and analyzed by 32-plex Luminex array after 3 days. D. Inhibitory receptors expression in CART19 cells in the presence TP0903. UTDs or CART19 were incubated with Jeko at a 1 to 1 E/T ratio in the presence of TP0903. At day 5, T cells were stained with PD-1 (left), LAG-3 (right), and TIM-3. E. CART19 cytotoxicity in the presence TP0903: CART19 or UTDs were co-cultured at different effector-to-target ratios (E:T) with luciferase-positive Jeko in the presence of increasing doses of TP0903. Cell killing was assessed by luminescence at 24 hours. F. TP0903 enhanced CART cell persistence in vivo. NSG mice were injected with 1.0×10⁶ luciferase-positive JeKo on day −7. At day 0, mice were randomized according to tumor burden to receive vehicle, TP0903 20 mg/kg/day, 1.0×10⁶ CART19, or TP0903 20 mg/kg/day+0.5×10⁶ CART19. At day 21, mice were re-challenged with JeKo.

FIG. 20. Multiparametric flow cytometric analysis of peripheral blood samples obtained before and after TP0903 treatment in patients with advanced solid tumors. A. Representative CD4⁺CD25⁺Foxp3⁺ regulatory T cells (Treg) (upper right quadrants) at baseline (left) and after treatment (right). Data were pooled from three independent experiments with peripheral blood mononuclear cells from three donors. B. Representative flow plot of CD4⁺ and CCR6⁻ negative cells. Remarked reduction of CXCR3⁻CCR4⁺ fraction (Th2) and increase of CXCR3⁺CCR4⁺ (double positive) fraction after TP0903 treatment.

FIG. 21A-E. TP0903 modulates naïve T cells function upon stimulation of PMA/Ionomycin in vitro. A. Naïve T cell proliferation assay in the presence of TP0903. Naïve T cells were stimulated with 5 ng/ml of PMA and 0.1 μg/ml of Ionomycin in the absence or in the increasing dose of TP0903 (10-65 nM) for 3 days. TP0903 did not interfere T cell expansion. B. At the end of the expansion on day 3, T cells were stained with inhibitory receptors. In the presence of high dose TP0903, inhibitory receptors (PD-1, LAG-3, TIM-3, and ICOS) were significantly downregulated. C. Naïve T cell degranulation assay and cytokine production in the presence of TP0903. Naïve T cells were stimulated with 50 ng/ml of PMA and 1 μg/ml of Ionomycin for 4 hours in the presence of increasing doses of TP0903 (10-65 nM). Flow cytometric analysis revealed similar activation of T cells, in the presence of TP0903 as shown by CD107a degranulation and intracytoplasmic cytokine production (IL2, IFN-g, and GM-CSF). However, there was a significant inhibition of IL-4 in the increasing dose of TP0903. D. Western blot analysis of p-LCK and GATA-3 inhibition by TP0903 in naïve T cells. Naïve T cells were stimulated with 5 ng/ml of PMA and 0.1 ug/ml of Ionomycin in the absence (DMSO) or in the increasing dose of TP0903 (10-65 nM) for 24 hours. Western blot analysis on protein lysates revealed modest inhibition of the phosphorylation of LCK and GATA-3 at the highest dose (65 nM). 3-actin was used as loading control. E. TP0903 specifically reduces regulatory T cells (Treg). Effector T cells (Teff) were isolated by negative selection using RoboSep™ Human T Cell Isolation Kit (Stemcell Technologies). Tregs were isolated by negative selection using the human CD4⁺CD127^{low}CD25⁺ Treg isolation kit (Stemcell Technologies). Teff and Treg were stimulated with anti-CD3/CD28 beads and 400 U/ml of IL-2. At day 5, total T cells were evaluated via CountBright quantification. Teff expansion was not interfered by TP0903. However, Treg expansion was specifically reduced by TP0903.

FIG. 22A-C. In vitro experiment for the combination of CART19 and TP0903. A. CART19 were cocultured at different effector-to-target ratio (E:T) with luciferase-positive JeKo-1. At 48 hours, cell killing was assessed by luminescence. At a specific E:T ratio, increased cytotoxic effect was significantly correlated to increased TP0903 dose. The means and SEM are shown (**, p<0.005, *, p<0.05, one-way ANOVA analysis). n.s., not statistically significant. B. CART19 degranulation assay and cytokine production in the presence of TP0903. CART19 were cocultured with JeKo-1 for 4 hours in the presence of TP0903 doses of TP0903 (10-65 nM). Flow cytometric analysis revealed significant activation of CART19 cells upon stimulation of JeKo-1 cell lines in the presence or absence of TP0903 as shown by CD107a degranulation and intracytoplasmic cytokine production (IL-2, IFN-γ, GM-CSF). On the other hand, secretion of IL-4 was downregulated in the increasing dose of TP0903. C. Th2/Th1 T cell subtype analysis during CART19 expansion in the presence pf TP0903. CART19 cells were stimulated with lethally irradiated (120 Gy) JeKo-1 cell lines in the presence of increasing doses of TP0903 (10-30 nM). At the day 3, Th1 and Th2 cells were defined using CD4, CCR6, CCR4 and CXCR3 expression. There was a major shift toward Th1 phenotype. The means and SEM are shown (*, p<0.05, one-way ANOVA analysis). n.s., not statistically significant.

FIG. 23. CART19 cytokine production in the presence of TP0903. CART19 cells or untransduced T cells were co-cultured with JeKo-1 cells or leukemic B cells derived from patients with chronic lymphocytic leukemia for 3 days and supernatants were analyzed for human cytokines (Luminex, 38-plex). The cytokine production ability of IFN-γ, GM-CSF, TNF-α, and IL-2 was maintained in the presence of TP0903, however, Th2 related cytokines, such as IL-4 and IL-5 were significantly downregulated. Cytokines known as cytokine related syndrome, such as IL-6, IL-10, and MIP1-β were also remarkably downregulated in the presence of TP0903.

FIG. 24A-B. A. Western blot analysis of p-LCK inhibition by TP0903 in CART19 cells. CART19 cells were incubated with lethally irradiated JeKo-1 in the presence of TP0903 at a 1 to 3 ratio for 24 hours. After the incubation, CART19 cells were purified with anti-CD19 micro beads. Western blot analysis on protein lysates revealed modest inhibition of the phosphorylation of LCK and p38MAPK at the highest dose (65 nM). GATA-3 and T-bet were also inhibited at the highest dose. β-actin was used as loading control. B. Comparison of gene expression between TP0903 treated CART19 cells and non-treated CART19 cells subpopulations using RNA-seq. CART19 cells from three biological replicates were thawed and stimulated with irradiated JeKo-1 (120 Gy) for 5 days. Each sample was treated with either 30 nM TP0903 (treated condition) or DMSO (untreated condition). Representative flow cytometric analysis of CART19 cells before RNA isolation (right). RNA-seq was performed on an Illumina HTSeq 4000 by the Genomic Sequencing Core at Mayo Clinic.

FIG. 25A-E. Impact of the combinatory therapy in vivo. A. Schema for in vivo experiment. B and C. Combination therapy of CART19 and TP0903 showed superior low tumor burden to CART19 monotherapy. Moreover, combination arm successfully rejected re-challenging luciferase positive JeKo-1. D. Reduction of MCP-1, which is known as CRS related cytokine in the presence of TP0903. E. Expansion of circulating CART19 after re-challenging Luciferase positive JeKo-1.

FIG. 26A-C. A, Leukemic B cells derived from patients with chronic lymphocytic leukemia (red line) and JeKo-1 cells (green line) were stained with CD19, CD5, and mouse IgG1 kappa isotype control (solid lines) or AXL monoclonal antibody. B, CLL B cells overexpress Axl. Lysates from purified CLL B cells (P1-P4) and were examined for the expression of Axl by Western blot analysis using AXL antibody. Actin was used as a loading control. C, CART19 cells were stimulated with lethally irradiated JeKo-1 for 24 hours. After the co-culture, CART19 cells were isolated with CD19 microbeads. The dot plot demonstrated that CART19 cells were successfully isolated after the co-culture. UTD were stimulated with PMA and Ionomycin. Then, naïve T cells, stimulated UTD cells, unstimulated CART19 cells, and CART19 cells were analyzed for the expression of Axl by Western blotting. Though T cells expressed Axl regardless of stimulation or presence of CAR19, Axl expression was downregulated when CART19 cells were stimulated with CD19⁺ antigen.

FIG. 27. CART19 cytokine production in the presence of TP-0903. CART19 cells or untransduced T cells were co-cultured with JeKo-1 cells (left heat map) or leukemic B cells (right heat map) derived from patients with chronic lymphocytic leukemia for 3 days, and supernatants were analyzed for human cytokines (Luminex, 38-plex). The cytokine production ability of IFN-γ, GM-CSF, TNF-α, and IL-2 was maintained in the presence of TP-0903, however, Th2 related cytokines, such as IL-4 and IL-5 were significantly downregulated. Cytokines known as cytokine related syndrome, such as IL-6, IL-10, and MIP1-β, also were remarkably downregulated in the presence of TP-0903. IL-8 also was downregulated in the presence of TP-0903.

FIG. 28A-E. A, Western blot analysis of p-LCK inhibition by TP-0903 in CART19 cells. CART19 cells were incubated with lethally irradiated JeKo-1 in the presence of TP0903 at a 1 to 3 ratio for 24 hours. After the incubation, CART19 cells were purified with anti-CD19 micro beads. B, Representative flow cytometric analysis of CART19 cells before RNA isolation. Principal component analysis (PCA) from RNA-seq analysis of untreated cells and cells treated with 30 nM of TP-0903. C, Volcano plot analysis indicates genes with increased expression in TP-0903 treated CART19 cells (right) or decreased expression in TP-0903 treated CART19 cells (left). D, Comparison of gene expression between TP treated CART19 cells and non-treated CART19 cells subpopulations using RNA-seq. CART19 cells from three biological replicates were thawed and stimulated with irradiated JeKo-1 (120 Gy) for 5 days. Each sample was treated with either 30 nM TP-0903 (treated condition) or DMSO (untreated condition). RNA-seq was performed on an Illumina HTSeq 4000. E, This table shows gene ontology for significantly upregulated genes in CART19 treated with TP-0903 and biological processes that overlap with the significantly differentially upregulated genes using Enrichr.

### DETAILED DESCRIPTION

This document provides methods and materials involved in treating cancer. For example, one or more TAM RTK inhibitors (e.g., one or more AXL inhibitors) can be administered in combination with an adoptive cell therapy (e.g., a CART cell therapy) to alter (e.g., enhance or suppress) one or more functions of a T cell and/or to enhance T cell expansion. In some cases, a composition including one or more TAM RTK inhibitors can be used to condition T cells (e.g., CART cells) ex vivo (e.g., for use in adoptive cell therapy). For example, a composition including one or more TAM RTK inhibitors can be administered to T cells (e.g., CART cells) ex vivo to alter (e.g., enhance or suppress) one or more functions of the T cell in adoptive T cell therapies (e.g., CART cell therapies) and/or to enhance T cell expansion. In some cases, a composition including one or more TAM RTK inhibitors can be used to alter (e.g., enhance or suppress) one or more functions of T cells (e.g., CART cells) in vivo. For example, a composition including one or more TAM RTK inhibitors can be administered to a mammal being treated with an adoptive cell therapy (e.g., a CART cell therapy) to alter (e.g., enhance or suppress) one or more functions of a T cell and/or to enhance expansion of a T cell within the mammal (e.g., a T cell administered to the mammal during the adoptive cell therapy procedure). In some cases, altering (e.g., enhancing or suppressing) one or more functions of a T cell (e.g., a CART cell) and/or enhancing T cell (e.g., CART cell) expansion as described herein can be used to reduce the number of cancer cells (e.g., cancer cells expressing a tumor antigen) within a mammal.

As described herein, one or more TAM RTK inhibitors (e.g., one or more AXL inhibitors) used in combination with CART cell therapy can be used to alter (e.g., enhance or suppress) one or more functions of a T cell (e.g., a CART cell). T cell functions can include, without limitation, Th cell differentiation, expression of one or more receptors (e.g., inhibitory receptors), release of cytokines (e.g., CRS critical cytokines), IL-6, MIP1b, and MCP. In some cases, a composition including one or more TAM RTK inhibitors can be used in combination with CART cell therapy to enhance Th cell differentiation in a mammal. For example, one or more TAM RTK inhibitors can be used in combination with CART cell therapy to promote Th2 cells to differentiate into Th1 cells in a mammal. In some cases, a composition including one or more TAM RTK inhibitors can be used in combination with CART cell therapy to suppress (e.g., reduce or eliminate) expression of inhibitory receptors on one or more CART cells. For example, one or more TAM RTK inhibitors can be used in combination with CART cell therapy to reduce or eliminate expression of programmed cell death protein 1 (PD1) receptors, T cell immunoglobulin and mucin-domain containing 3 (TIM3) receptors, lymphocyte activation gene 3 (LAG3) receptors, and/or CTLA-4 receptors on one or more CART cells. In some cases, a composition including one or more TAM RTK inhibitors can be used in combination with CART cell therapy to suppress (e.g., reduce or eliminate) one or more side effects of CART cell therapy (e.g., CRS, fevers, low blood pressure, and/or neurotoxicity). For example, one or more TAM RTK inhibitors can be used in combination with CART cell therapy to reduce or eliminate CRS by reducing or eliminating the release of one or more cytokines associated with CRS (e.g., MCP1, IL-6, IL-10, and MIP-b) by one or more CART cells.

One or more TAM RTK inhibitors (e.g., one or more AXL inhibitors) can be used in combination with an adoptive T cell therapy (e.g., a CART cell therapy) to treat a mammal having any appropriate cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can express a tumor-associated antigen (e.g., an antigenic substance produced by a cancer cell). Examples of cancers that can be treated as described herein include, without limitation, lymphoma (e.g., B cell lymphomas such as diffuse large cell lymphoma (DLBCL)), leukemia (e.g., chronic lymphocytic leukemia (CLL) and acute lymphoblastic leukemia (ALL)), germ cell tumors, hepatocellular carcinoma, bowel cancer, lung cancer, breast cancer, ovarian cancer, melanoma, multiple myeloma, acute myeloid leukemia, and pancreatic cancer. For example, one or more AXL inhibitors can be used in combination with CART cell therapy to treat a lymphoma. In another example, one or more AXL inhibitors can be used in combination with CART cell therapy to treat a leukemia.

One or more TAM RTK inhibitors (e.g., one or more AXL inhibitors) can be used in combination with an adoptive T cell therapy (e.g., a CART cell therapy) targeting any appropriate antigen within a mammal (e.g., a mammal having cancer). In some cases, an antigen can be a tumor-associated antigen (e.g., an antigenic substance produced by a cancer cell). Examples of tumor associated antigens that can be targeted by an adoptive T cell therapy provided herein include, without limitation, cluster of differentiation 19 (CD19; associated with B cell lymphomas, acute lymphoblastic leukemia (ALL), and chronic lymphocytic leukemia (CLL)), alphafetoprotein (AFP; associated with germ cell tumors and/or hepatocellular carcinoma), carcinoembryonic antigen (CEA; associated with bowel cancer, lung cancer, and/or breast cancer), CA-125 (associated with ovarian cancer), mucin 1 (MUC-1; associated with breast cancer), epithelial tumor antigen (ETA; associated with breast cancer), melanoma-associated antigen (MAGE; associated with malignant melanoma), CD33 (associated with AML), CD123 (associated with AML), CLL1 (associated with AML), HER2 (associated with breast or ovarian cancer), EGFR (associated with lung, ovarian, or colon cancer), EGFRviii (associated with brain cancer), IL13R (associated with brain cancer), EPhA3 (associated with brain cancer), BCMA (associated with myeloma), CS1 (associated with myeloma), CD38 (associated with myeloma), CD138 (associated with myeloma or AML), FAP (associated with different cancers), CALR (associated with myeloid cancer), Mesothelin (associated with mesothelioma or pancreatic cancer), MUC (associated with breast cancer), CD22 (associated with B cell malignancies), Kappa (associated with B cell malignancies), Lambda (associated with B cell malignancies), CD20 (associated with B cell malignancies), CD30 (associated with Hodgkin lymphoma), CD3 (associated with T cell leukemia/lymphoma) CD5 (associated with T cell leukemia/lymphoma), CD7 (associated with T cell leukemia/lymphoma), and CD2 (associated with T cell leukemia/lymphoma). For example, one or more AXL inhibitors can be used in combination with CART cell therapy targeting CD19 (e.g., CART19 cell therapy) to treat cancer as described herein.

Any type of mammal having a cancer can be treated as described herein. For example, humans and other primates such as monkeys having a cancer can be treated with a composition including one or more TAM RTK inhibitors (e.g., one or more AXL inhibitors) in combination with an adoptive T cell therapy (e.g., a CART cell therapy). In some cases, dogs, cats, horses, cows, pigs, sheep, rabbits, mice, and rats can be treated as described herein. In some cases, a mammal can be identified as having cancer. Any appropriate method can be used to identify a mammal as having cancer. Once identified as having cancer, the mammal can be administered, or instructed to self-administer, one or more TAM RTK inhibitors (e.g., one or more AXL inhibitors) described herein (as a combined treatment with adoptive T cell therapy such as CAR-T therapy).

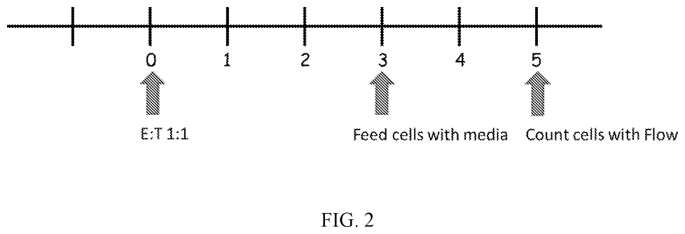

Any appropriate TAM RTK inhibitor can be used as described herein (as a combined treatment with adoptive T cell therapy such as CAR-T therapy). In some cases, a TAM RTK inhibitor such as an AXL inhibitor can be used as described herein. An AXL inhibitor can be an inhibitor of AXL polypeptide expression or an inhibitor of AXL polypeptide activity. Examples of AXL inhibitors (e.g., small molecule compounds) that reduces AXL polypeptide activity that can be used as described herein include, without limitation, TP0903, foretinib, cabozantinib, merestinib, MGCD265, ASLAM002, NPS-1034, LDC1267, bosutinib, giltertinib, SGI-7079, crizotinib, amuvatinib, UNC2025, 549076, sunitinib, and BGB324. See also, FIGS. 2, 4, 5, 6, 7, 8, and 9 of the Myers et al. reference (J. Med. Chem., 59(8):3593-3608 (2016)), which are hereby incorporated by reference herein. Examples of compounds that reduce AXL polypeptide expression that can be used as described herein include, without limitation, nucleic acid molecules designed to induce RNA interference (e.g., an RNAi molecule or a siRNA molecule), antisense molecules, and miRNAs. AXL inhibitors can be readily designed based upon the nucleic acid and/or polypeptide sequences of AXL. Examples of AXL nucleic acids include, without limitation, the human AXL sequence set forth in GenBank® Accession No. NM_001699 (Version No. NM_001699.5) and M76125 (Version No. M76125.1). Examples of AXL polypeptides include, without limitation, the human AXL polypeptide having the amino acid sequence set forth in GenBank® accession number EAW57023 (Version No. EAW57023.1) and AAH32229 (Version No. AAH32229.1).

TP0903 refers to a compound of structure (I) shown below, or a pharmaceutically acceptable salt thereof:

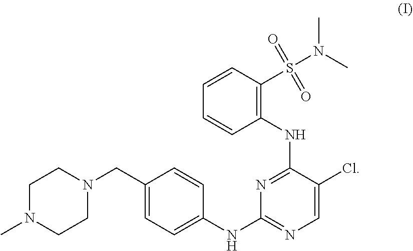

See also U.S. Pat. No. 8,901,120.

In some cases, one or more (e.g., one, two, three, four, five, or more) TAM RTK inhibitors (e.g., one or more AXL inhibitors) can be administered in combination with an adoptive T cell therapy (e.g., a CART cell therapy) to a mammal (e.g., a mammal having cancer) to alter (e.g., enhance or suppress) one or more functions of the CART cell and/or to enhance expansion of the CART cell within the mammal. For example, two or more AXL inhibitors can be administered to a mammal having cancer (e.g., a human having cancer) to alter (e.g., enhance or suppress) one or more functions of a CART cell and/or to enhance expansion of a CART cell within the mammal.

One or more TAM RTK inhibitors (e.g., one or more AXL inhibitors) used in combination with an adoptive T cell therapy (e.g., a CART cell therapy) can be administered in any appropriate order with respect to the administration of the adoptive T cell therapy. In some cases, a composition including one or more TAM RTK inhibitors can be administered concurrently with the administration of the adoptive T cell therapy. For example, in cases where one or more TEM RTK inhibitors are administered to a mammal (e.g., in vivo administration), a composition including one or more AXL inhibitors can be administered to the mammal any time during the course of an adoptive T cell therapy procedure. In some cases, a composition including one or more TAM RTK inhibitors can be administered in series with the administration of the adoptive T cell therapy. For example, in cases where one or more TEM RTK inhibitors are administered to a mammal (e.g., in vivo administration), a composition including one or more AXL inhibitors can be administered before, after, or both before and after the adoptive T cell therapy procedure. In some cases, a composition including one or more AXL inhibitors can be administered before, during, and after the adoptive T cell therapy procedure.

In some cases, one or more TAM RTK inhibitors can be used ex vivo. For example, a composition including one or more TAM RTK inhibitors can be administered to T cells (e.g., CART cells) of an adoptive T cell therapy procedure ex vivo. In some cases, T cells of an adoptive T cell therapy procedure can be contacted with a composition including one or more TAM RTK inhibitors at the time the T cells are being administered a CAR (or a compound expressing a CAR). For example, T cells can be exposed to nucleic acid encoding a CAR and a composition including one or more TAM RTK inhibitors to produce T cells for an adoptive T cell therapy procedure.

One or more TAM RTK inhibitors (e.g., one or more AXL inhibitors) used in combination with an adoptive T cell therapy (e.g., a CART cell therapy) can be administered to a mammal having a cancer as a combination therapy with one or more additional agents used to treat a cancer. For example, one or more TAM RTK inhibitors used in combination with an adoptive cell therapy can be administered to a mammal in combination with one or more anti-cancer treatments (e.g., radiation therapy, chemotherapy, targeted therapies, hormonal therapy, angiogenesis inhibitors, immunotherapy, checkpoint blockade, immunomodulatory agents, IDO inhibitors, and/or immune stimulants). In cases where a composition including one or more TAM RTK inhibitors used in combination with an adoptive cell therapy is used with additional agents treat a cancer, the one or more additional agents can be administered at the same time or independently. In some cases, a composition including one or more TAM RTK inhibitors used in combination with an adoptive cell therapy can be administered first, and the one or more additional agents administered second, or vice versa.

In some cases, one or more TAM RTK inhibitors (e.g., one or more AXL inhibitors) used in combination with an adoptive T cell therapy (e.g., a CART cell therapy) can be formulated into a pharmaceutically acceptable composition for administration to a mammal having cancer. For example, a therapeutically effective amount of a TAM RTK inhibitor can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol such as Vitamin E TPGS, sodium carboxymethylcellulose, polyacrylates, waxes, polyethylene-polyoxypropylene-block polymers, and wool fat.

A composition (e.g., a pharmaceutical composition) containing one or more TAM RTK inhibitors (e.g., one or more AXL inhibitors) used in combination with an adoptive T cell therapy (e.g., a CART cell therapy) can be designed for oral or parenteral (including subcutaneous, intramuscular, intravenous, and intradermal) administration. When being administered orally, a pharmaceutical composition containing one or more TAM RTK inhibitors can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more TAM RTK inhibitors (e.g., one or more AXL inhibitors) used in combination with an adoptive T cell therapy (e.g., a CART cell therapy) can be administered locally or systemically. For example, a composition containing one or more TAM RTK inhibitors can be administered systemically by an oral administration or by injection to a mammal (e.g., a human).

Effective doses of one or more TAM RTK inhibitors (e.g., one or more AXL inhibitors), used, for example, in combination with adoptive T cell therapy, can vary depending on the severity of the cancer, the route of administration, the age and general health condition of the subject, excipient usage, the possibility of co-usage with other therapeutic treatments such as use of other agents, and the judgment of the treating physician.

An effective amount of a composition containing one or more TAM RTK inhibitors (e.g., one or more AXL inhibitors) can be any amount that alters (e.g., enhances or suppresses) one or more functions of a CART cell and/or enhances CART cell expansion without producing significant toxicity to the mammal. An effective amount of an AXL inhibitor such as TP0903 can be from about 10 nM to about 500 nM (e.g., from about 10 nM to about 100 nM, from about 20 nM to about 100 nM, from about 30 nM to about 100 nM, from about 40 nM to about 100 nM, from about 50 nM to about 100 nM, from about 60 nM to about 100 nM, from about 70 nM to about 100 nM, from about 80 nM to about 100 nM, from about 90 nM to about 100 nM, from about 10 nM to about 90 nM, from about 10 nM to about 80 nM, from about 10 nM to about 70 nM, from about 10 nM to about 60 nM, from about 10 nM to about 50 nM, from about 10 nM to about 40 nM, from about 10 nM to about 30 nM, from about 10 nM to about 20 nM, from about 20 nM to about 90 nM, from about 30 nM to about 80 nM, from about 40 nM to about 70 nM, from about 50 nM to about 60 nM, from about 30 nM to about 50 nM, from about 50 nM to about 70 nM, or from about 60 nM to about 80 nM). For example, an effective amount of TP0903 can be about 10 nM. In some cases, an effective amount of TP0903 can be about 30 nM. For example, an effective amount of TP0903 can be about 65 nM. For example, an effective amount of TP0903 can be about 100 nM. In some cases, a low dose (e.g., about 10 nM) of TP0903 can be an effective amount. In some cases, a high dose (e.g., about 100 nM) of TP0903 can be an effective amount. In some cases, the TP0903 can be administered to a mammal, in combination with CAR-T cells, at a daily dose of about 1-37 mg/m² (e.g., about 1-25 mg/m²) or at a daily dose of about 1-75 mg (e.g., about 1-50 mg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., a cancer) may require an increase or decrease in the actual effective amount administered.

The frequency of administration of a composition containing one or more TAM RTK inhibitors (e.g., one or more AXL inhibitors) can be any frequency that alters (e.g., enhances or suppresses) one or more functions of a CART cell and/or enhances CART cell expansion without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing one or more TAM RTK inhibitors (e.g., one or more AXL inhibitors) can include rest periods. For example, a composition containing one or more TAM RTK inhibitors (e.g., one or more AXL inhibitors) can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., a cancer) may require an increase or decrease in administration frequency.

An effective duration for administering a composition containing one or more TAM RTK inhibitors (e.g., one or more AXL inhibitors) can be any duration that alters (e.g., enhances or suppresses) one or more functions of a CART cell and/or enhances CART cell expansion without producing significant toxicity to the mammal. For example, the effective duration can vary from several days to several weeks, months, or years. In some cases, the effective duration for the treatment of a cancer can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In some cases, one or more functions of a CART cell and/or CART cell expansion can be monitored. Any appropriate method can be used to determine whether or not one or more functions of a CART cell are altered (e.g., enhanced or suppressed) and/or whether or not CART cell expansion is enhanced at different time points. Examples of methods that can be used to evaluate CART cell functions include, without limitation, cytotoxicity assays (e.g., to evaluate whether or not CART cells are effective at killing target cells), cytokine assays (e.g., to evaluate Th cell differentiation and/or to evaluate whether or not cytokines related to CRS are being produced), Th polarization assays (e.g., to evaluate Th cell differentiation), and exhaustion assays (e.g., to evaluate whether or not expression of inhibitory molecules is suppressed). Examples of methods that can be used to evaluate CART cell expansion include, without limitation, proliferation assays (e.g., to evaluate the number of CART cells).

In some cases, the number of cancer cells present within a mammal, and/or the severity of one or more symptoms related to the cancer being treated can be monitored. Any appropriate method can be used to determine whether or not the number of cancer cells present within a mammal is reduced. For example, imaging techniques can be used to assess the number of cancer cells present within a mammal.

A composition containing one or more TAM RTK inhibitors (e.g., one or more AXL inhibitors) can be combined with packaging material and configured into a kit. The packaging material included in a kit can contain instructions or a label describing how the composition can be used, for example, in combination with an adoptive cell therapy (e.g., CART cell therapy) to alter (e.g., enhance or suppress) one or more functions of a CART cell as described herein and/or to enhance CART cell expansion as described herein. In some cases, a kit also can include materials for use in an adoptive cell therapy (e.g., CART cell therapy) procedure.

The invention will be further described in the following examples, which do not limit the scope of the invention described in the claims.

### EXAMPLES

### Example 1: Combination of CAR-T Cells with AXL Inhibitor Alters T Cell Functions and Prevents T Cell Exhaustion

AXL is a member of the TAM (TYRO3, AXL and Mer) subfamily of receptor tyrosine kinase (RTK). AXL is expressed within cellular components of the tumor microenvironment where AXL signaling contributes to the immunosuppressive and protumorigenic phenotypes. AXL plays important roles in cell inflammation, proliferation, survival and migration via different signaling pathways. AXL is not expressed on T cells.

### Methods

### Cell Lines and Primary Samples

Cell lines were obtained from ATCC (K-562, NALM6 and JEKO-1). All cell lines were tested for sterility before experiments. For some experiments, JEKO-1 cell and NALM-6 cells were transduced with zsGreen/GFP lentivirus and then sorted to obtain >99% positive population. Primary B cells were obtained from the Mayo Clinic CLL biobank. These samples were prospectively collected and cryopreserved. Cell lines MOLM-14 and K562 were used as controls as indicated in the relevant figures. The cell lines were maintained in culture with RPMI1640 (Gibco, 11875-085, LifeTechnologies) supplemented with 10% FBS (Gemini, 100-106) and 50 U/mL penicillin/streptomycin (Gibco, Life Technologies, 15070-063). For all functional studies, primary cells were thawed at least 12 hours before experiment and rested at 37° C.

### IHC

Thin-layer cell preparation was obtained by Cytospin (Thermo Scientific) and stained with Giemsa. For formalin-fixed paraffin-embedded tissues, immunohistochemical staining was performed on a Leica Bond-III instrument (Leica Biosystems) using the Bond Polymer Refine Detection System. Antibodies were used undiluted. Heat-induced epitope retrieval was done for 20 minutes with ER2 solution (Leica Microsystems, AR9640). Images were digitally acquired using the Aperio ScanScope (Leica Biosystems).

### Generation of CAR Constructs and CAR T Cells

The murine anti-CD19 chimeric antigen receptor (single chain variable fragment derived from clone FMC63, CD8 hinge, 4-1BB costimulatory domain and CD3 zeta signaling domain) was generated de novo and cloned into a third generation lentivirus. Normal donor T cells were positively selected using negative selection Kit (Stem Cell), and expanded in vitro using anti-CD3/CD28 Dynabeads (Invitrogen, Life Technologies, Grand Island, N.Y., USA, added on the first day of culture). T cells were transduced with lentiviral supernatant one day following stimulation at a multiplicity of infection (MOI) of 3. The anti-CD3/CD28 Dynabeads were removed on day 6 and T cells were grown in T cell media (X-vivo 15 media, human serum 5%, penicillin, streptomycin and glutamax). CART cells were then cryopreserved on day 8 for future experiments. Prior to all experiments, T cells were thawed and rested overnight at 37° C.

### TP0903

TP0903 was obtained from Tolero pharmaceutical. For in vitro experiments, TP0903 was dissolved in DMSO and diluted to the indicated concentrations. For in vivo experiment, TP0903 powder was dissolved in Vitamin E TPGS (vehicle) and administered to mice via oral gavage at the indicated concentrations.

### Multiparametric Flow Cytometry

Anti-human antibodies were purchased from Biolegend (San Diego, Calif., USA), eBioscience (San Diego, Calif., USA) or BD Biosciences (San Jose, Calif., USA). Cells were isolated from in vitro culture or from animals, washed once in phosphate-buffered saline supplemented with 2% fetal calf serum and stained at 4° C. after blockade of Fc receptors. For cell number quantitation, Countbright beads (Invitrogen) were used according to the manufacturer's instructions (Invitrogen). In all analyses, the population of interest was gated based on time gating, followed by forward vs side scatter characteristics, followed by singlet gating, and live cells were gated using Live Dead Aqua (Invitrogen). Surface expression of anti-CD19 CAR was detected by staining with an Alexa Fluor 647-conjugated goat anti-mouse F(ab′)2 antibody from Jackson Immunoresearch (West Grove, Pa., USA). Flow cytometry was performed on a four-laser analyzer (BD Canto-II). All analyses were performed using FlowJo X10.0.7r2.

### T Cell Degranulation and Intracellular Cytokine Assays

Briefly, T cells were incubated with target cells at a 1:5 ratio. After staining for CAR expression, antibodies against CD107a, CD28, CD49d and monensin were added at the time of incubation. After 4 hours, cells were harvested and stained for CD3, CD4, and Live Dead staining (Invitrogen). Cells were fixed and permeabilized (FIX & PERM Cell Fixation & Cell Permeabilization Kit, Life Technologies) and intracellular cytokine staining was then performed as indicated in the specific experiments.

### Proliferation Assays

T cells were washed and re-suspended at 1×10⁷/mL in 100 μL of phosphate-buffered saline and labeled with 100 μL of carboxyfluorescein succinimidyl ester (CFSE) 2.5 μM (Life Technologies) for 5 minutes at 37° C. The reaction was then quenched with cold R10, and the cells were washed three times. Targets were irradiated at a dose of 100 Gy. T cells were incubated at a 1:1 ratio with irradiated target cells for 120 hours. Cells were then harvested, stained for CD3, CAR and Live Dead aqua (Invitrogen), and Countbright beads (Invitrogen) were added prior to flow cytometric analysis.

### Cytotoxicity Assays

JEKO-Luc cells or primary B cells were used for cytotoxicity assay. In brief, targets were incubated at the indicated ratios with effector T cells for 4 or 16 hours. Killing was calculated either by bioluminescence imaging on a Xenogen IVIS-200 Spectrum camera (PerkinElmer, Hopkinton, Mass., USA) or by flow cytometry. For the latter, cells were harvested; Countbright beads and life/dead (Invitrogen) were added prior to analysis. Residual live target cells were CD19+L/D−.

### Secreted Cytokine Measurement

Effector and target cells were incubated at a 1:1 ratio in T cell media for 24 or 72 hours as indicated. Supernatant was harvested and analyzed by 30-plex Luminex array according to the manufacturer's protocol (Millipore).

### In Vivo Experiments

NOD-SCID-γ chain−/− (NSG) originally obtained from Jackson Laboratories were maintained in our laboratory under an IACUC approved breeding protocol. Schemas of the utilized xenograft models are discussed in detail in the relevant figures and the Results section. Cells were injected in 200 μL of phosphate-buffered saline at the indicated concentration into the tail veins of mice. Bioluminescent imaging was performed using a Xenogen IVIS-200 Spectrum camera. Images were acquired and analyzed using Living Image version 4.4 (Caliper LifeSciences, Inc., PerkinElmer).

### Statistical Analysis

All statistical analyses were performed as indicated using GraphPad Prism 6 for Windows, version 6.04. Student t test was used to compare two groups; in analyses where multiple groups were compared, one-way ANOVA was performed with Holm-Sidak correction for multiple comparisons. When multiple groups at multiple time points/ratios were compared, the Student t test or ANOVA for each time points/ratios was used. Survival curves were compared using the log-rank test. In the figures, asterisks represent P values (*, P<0.05; **, P<0.01; ***, P<0.001; ****, P<0.0001) and “ns” means “not significant” (P>0.05). Further details of the statistics for each experiment are listed in figure legends.

### Results

### The Combination of CART19 and TP0903 is Synergistic

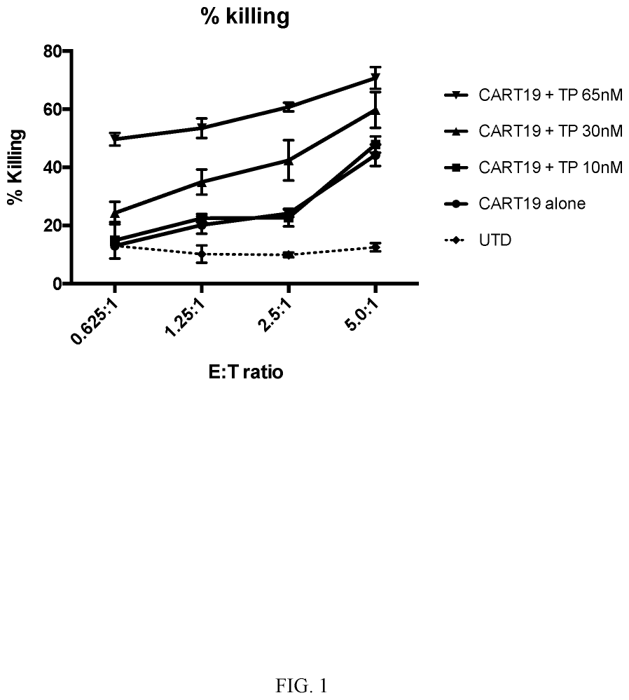

CART19 or UTDs were cocultured at different effector-to-target ratio (E:T) with luciferase-positive Jeko or with primary CLL cells, with increasing dose of TP0903 as indicated on the X axis. At 24 hours, cell killing was assessed by luminescence or by flow cytometry. The combination therapy of CART19 and TP0903 group showed robust killing at all E:T ratios (FIG. 1). Calculations suggest a synergistic rather than an additive effect.

### CART19 Proliferation in the Presence TP0903

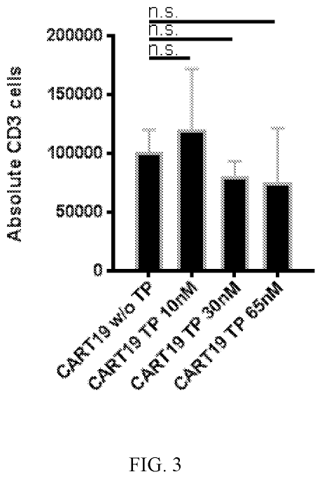

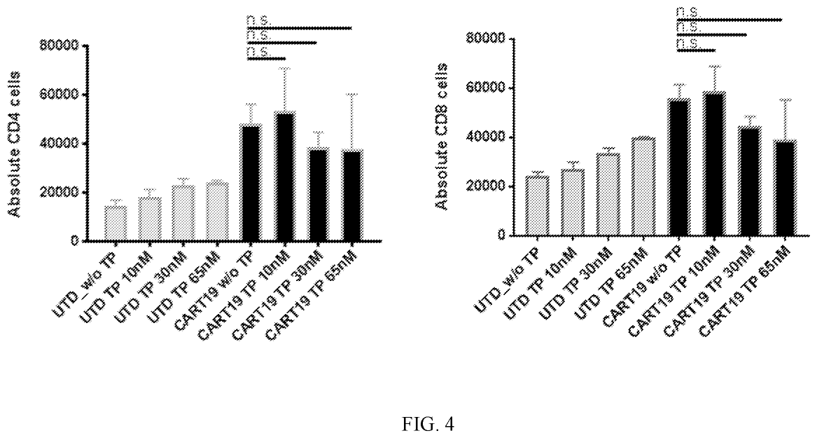

UTDs or CART19 were incubated with Jeko cell line or primary CLL cells in the presence of TP0903, at 1:1 ratio for 5 days. At the end of the experiment, cells were harvested and stained for CD3, live dead staining was performed, and counti bright beads were added prior to flow cytometric analyses (FIG. 2). Absolute T cell numbers were calculated based on this. The addition of TP0903 did not impair CART cell specific proliferation (FIG. 3 and FIG. 4). Low doses of TP0903 (10 nM) increased the number of CART19 cells. A high dose of TP0903 (>100 nM) decreased the number of CART19.

### T Cell Degranulation and Intracellular Cytokine Assays

T cells were incubated with target cells at a 1:5 ratio (experiments were done using JEKO cells as well as primary B cells). Antibodies against CD107a, CD28, CD49d and monensin were added at the time of incubation.

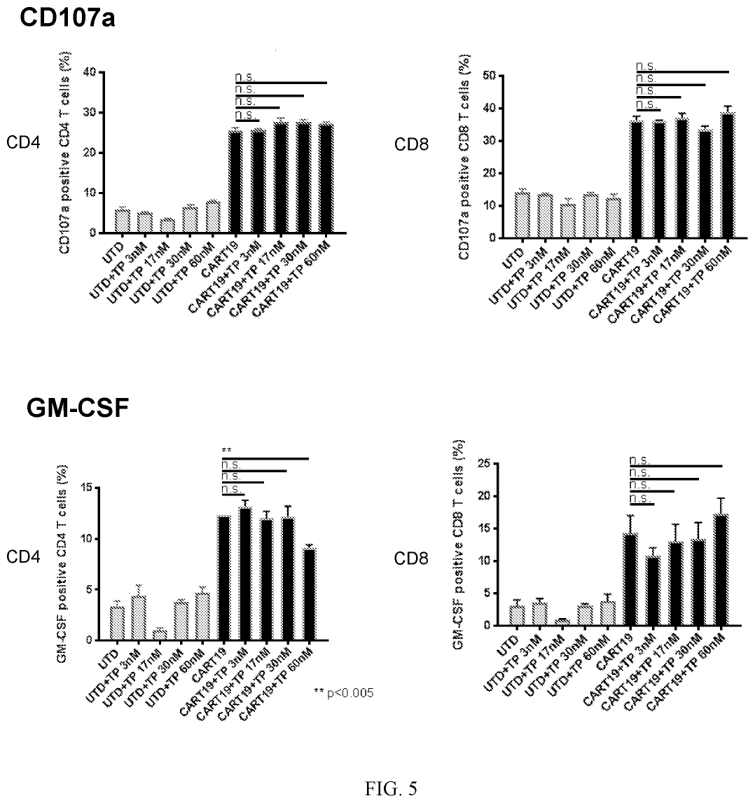

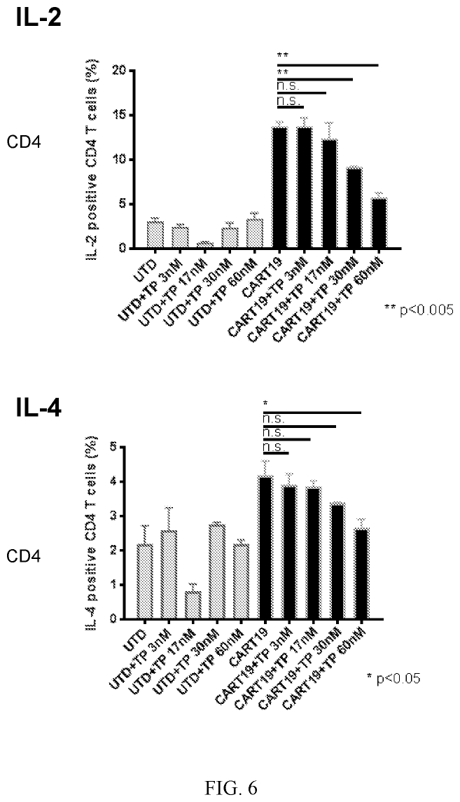

After 4 hours, cells were harvested and stained for CD3, CD4, and Live Dead staining (Invitrogen). Cells were fixed and permeabilized (FIX & PERM Cell Fixation & Cell Permeabilization Kit, Life Technologies), and intracellular cytokine staining was then performed. The addition of TP0903 did not impair CART cell functions, but lowered Th2 cytokines, suggesting that TP0903 differentiated T cells from Th2 into Th1 phenotype. The number of CD4 and CD8 cells positively stained for cluster of differentiation 107a (CD107a) or granulocyte-macrophage colony-stimulating factor (GM-CSF) in degranulation assays were shown in FIG. 5. The number of CD4 cells positively stained for interleukin-2 (IL-2) or interleukin-4 (IL-4) in degranulation assays were shown in FIG. 6.

### CART19 Cytokine Release in the Presence TP0903

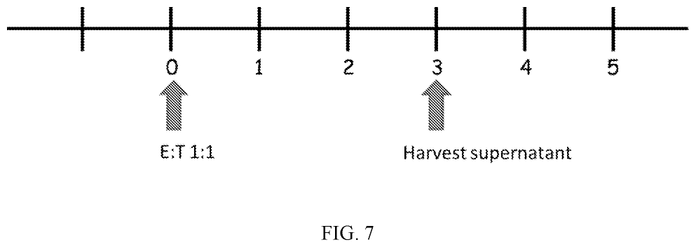

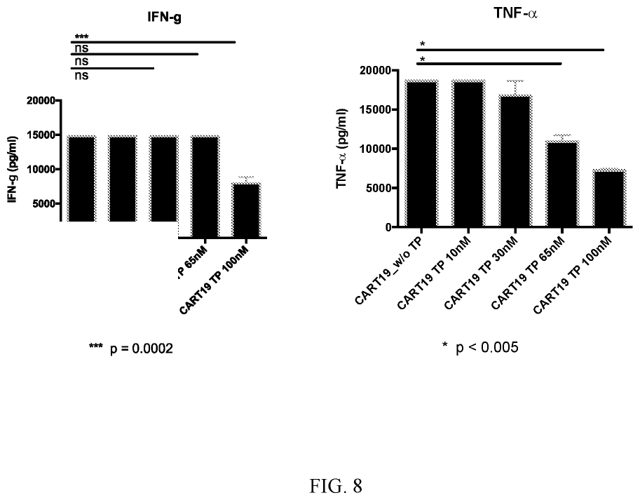

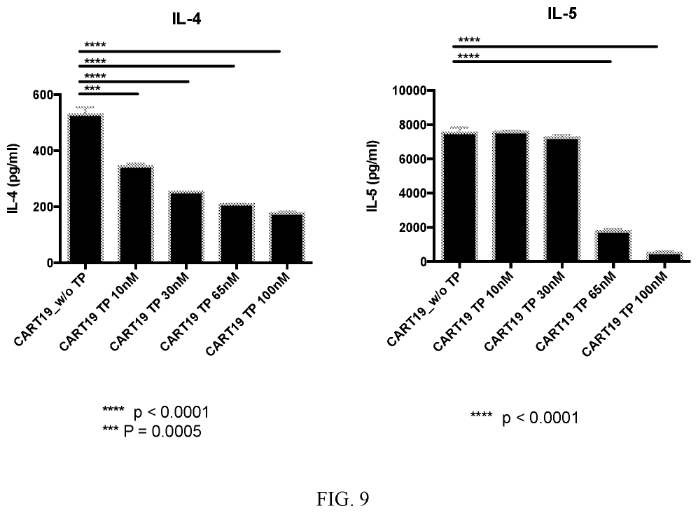

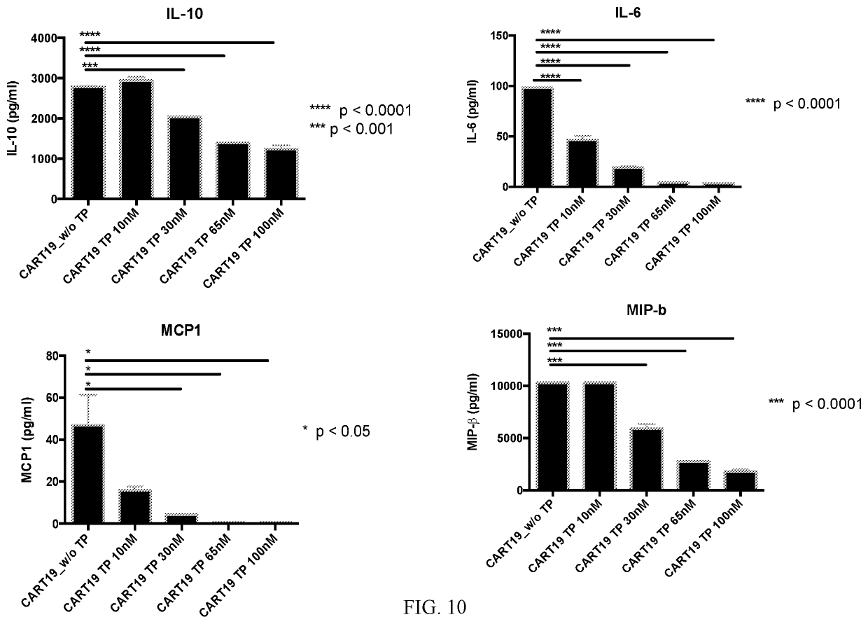

UTDs or CART19 were incubated with Jeko cell line or primary CLL cells in the presence of TP0903, at 1:1 ratio for 3 days (FIG. 7). At the end of the experiment, supernatant was harvested and analyzed for cytokines by 30-plex multiplex. TP0903 suppressed Th2 cytokines (FIG. 9), and only high doses (supra-physiological) suppressed Th1 cytokines (FIG. 8). TP0903 suppressed critical cytokines for the development of CRS after CART cell therapy (FIG. 10).

### T Cell Exhaustion

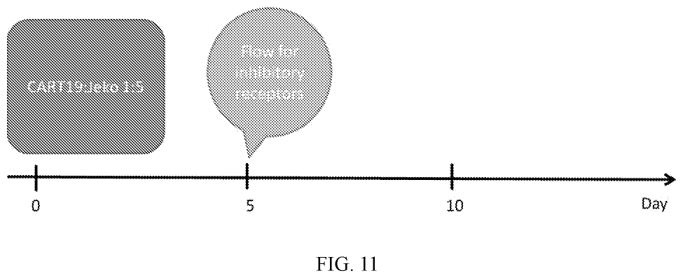

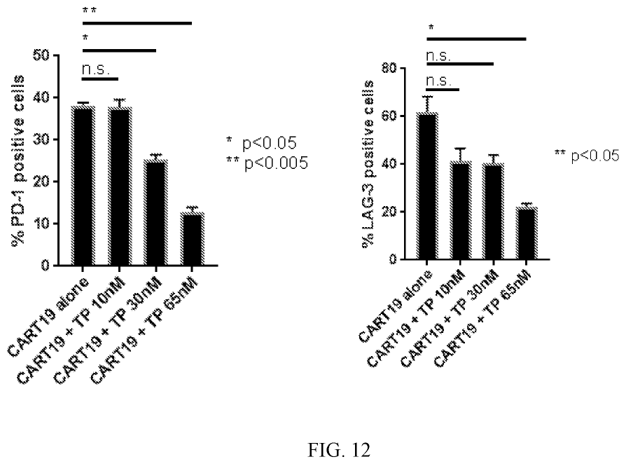

In vitro CART19 exhaustion assays in the presence of TP0903 were performed as shown in FIG. 11. T cells were incubated with target cells at a 1:5 ratio (experiments were done using JEKO cells as well as primary B cells). The low E:T ratio was used to induce a state of dysfunction of CART cells. Cells were cultured in the presence of increasing doses of TP0903. On day 5 of co-culture, cells were harvested, and expression of inhibitory receptors was analyzed. Co-culture in the presence of TP0903 reduced expression of PD1, TIM3, and LAG3 inhibitory receptors. TP0903 reduced exhaustion receptors on CART cells (FIG. 12). Inhibitory molecules were downregulated in a dose depended manner.

### TP0903 Enhances T Cell Expansion and CART Generation Ex Vivo

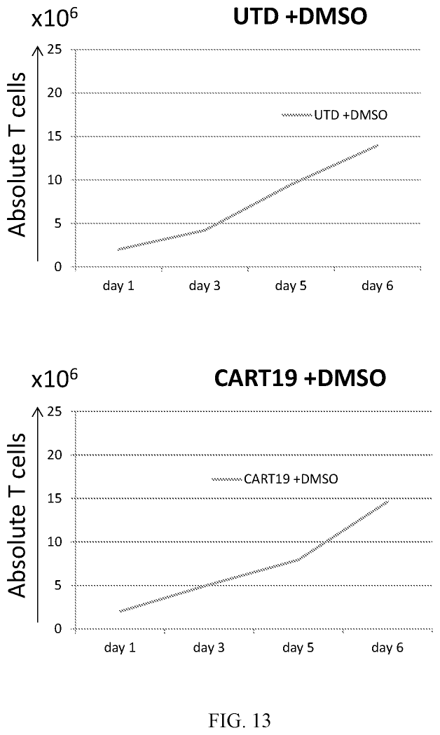

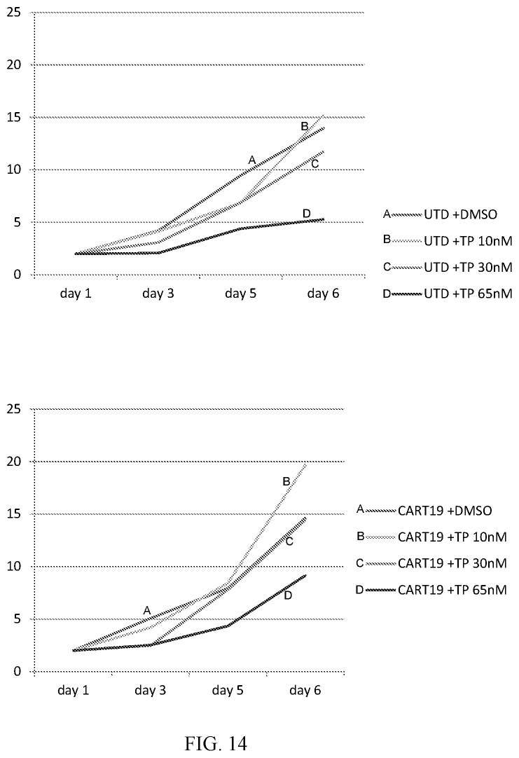

CART cells were generated with increasing doses of TP0903. T cells were isolated (negative selection, STEM CELL kit) and stimulated with CD3/CD28 beads on day 0. On day 1, T cells were transduced with CAR19 lentivirus at MOI of 3. Magnetic beads were removed on Day 6, and CART cells were frozen on day 8. Low doses of TP0903 enhanced T cell expansion ex vivo and CART cell generation (FIG. 13 and FIG. 14).

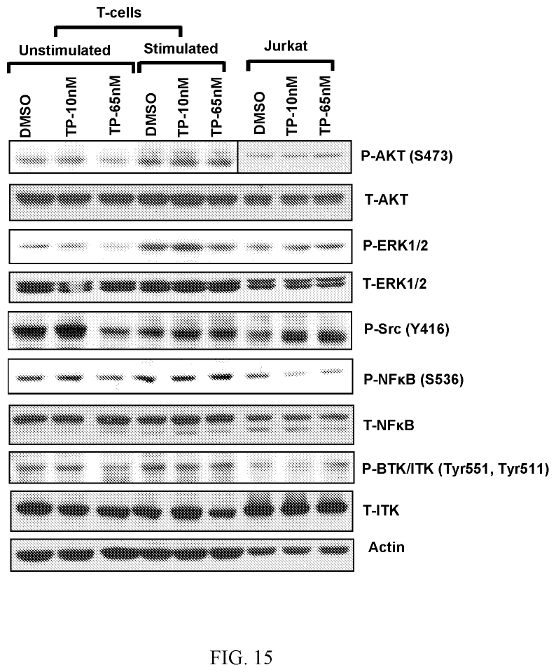

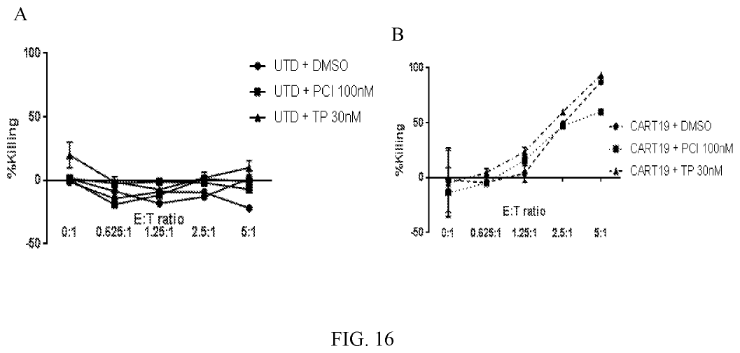

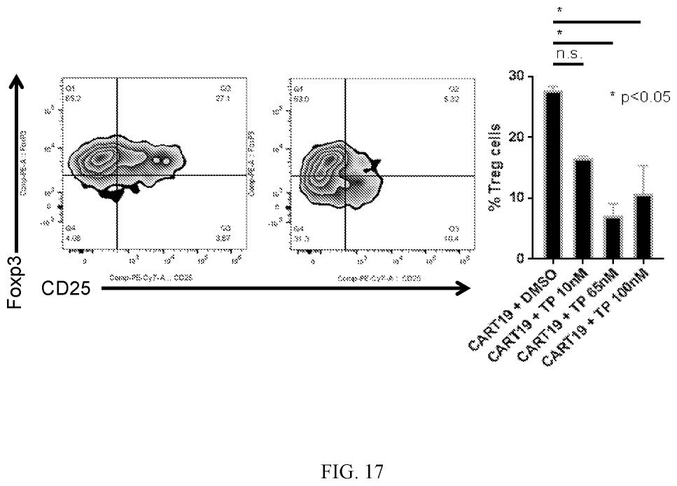

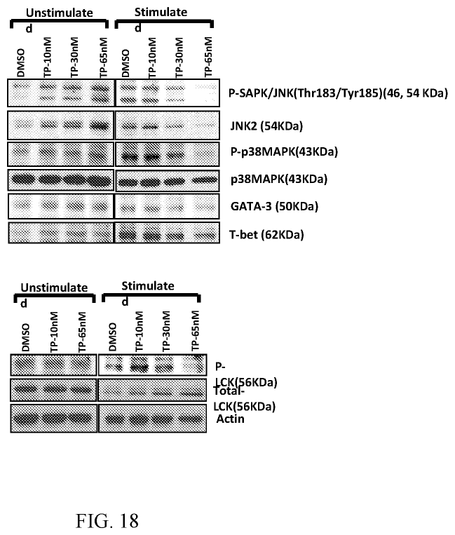

Additional results were provided in FIGS. 15-18.

### Example 2: Axl-RTK Inhibition Modulates T Cell Functions and Synergizes with Chimeric Antigen Receptor T Cell Therapy in B Cell Malignancies

This example examined the role of AXL RTK inhibition with TP0903 on T cell function in CLL and other B cell malignancies.

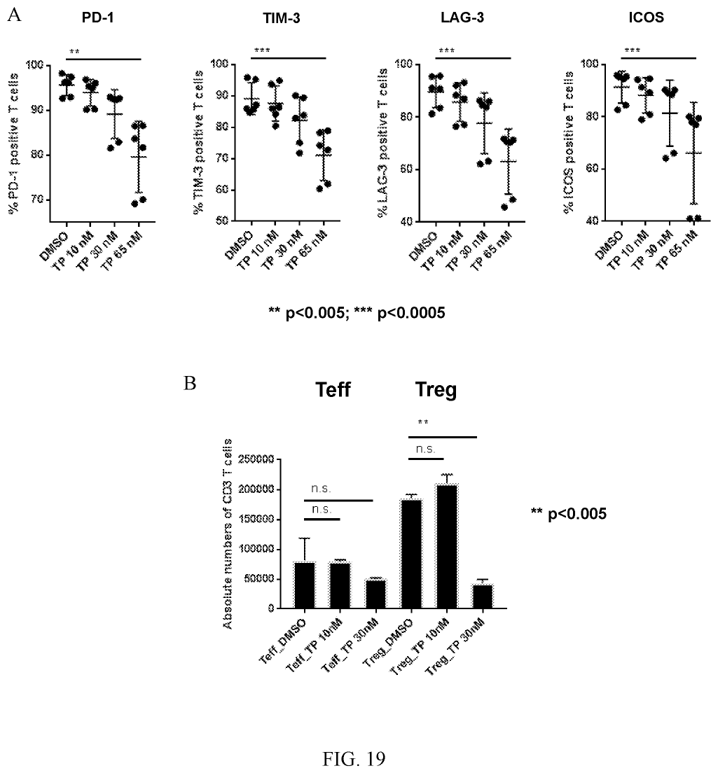

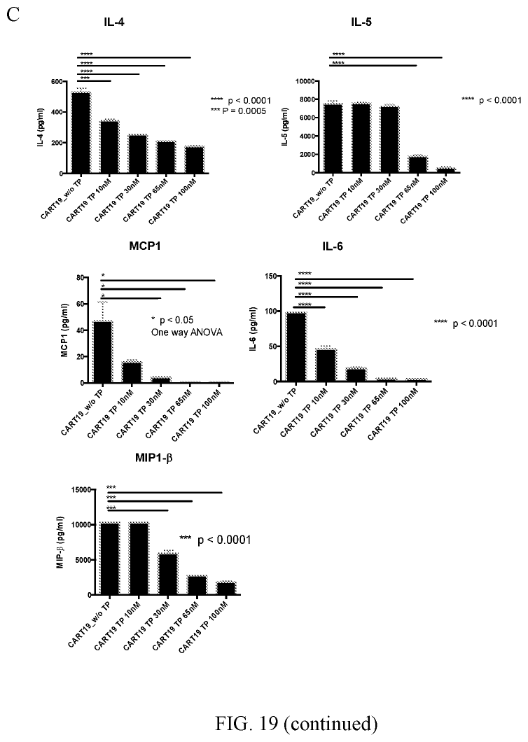

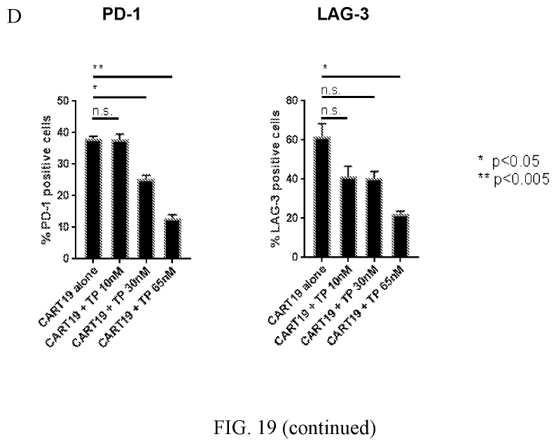

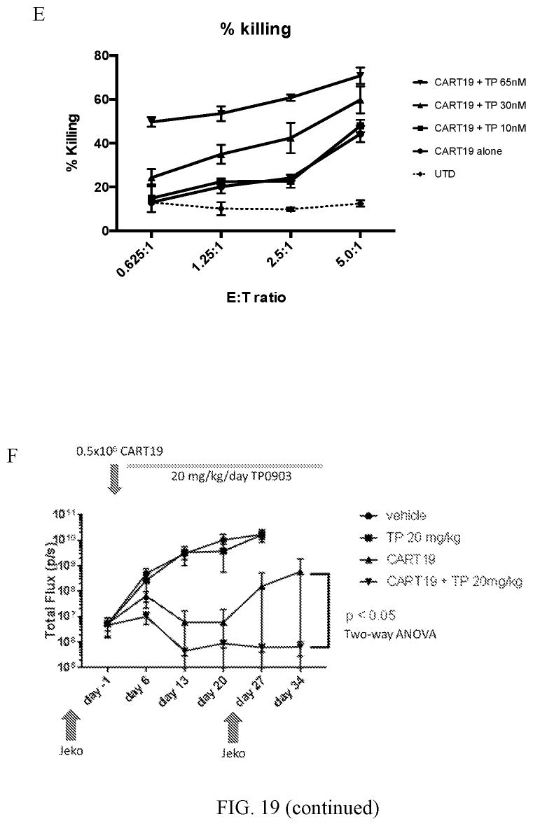

The effect of AXL inhibition on T cell phenotype in normal donors was investigated. When naïve T cells were stimulated with PMA/Ionomycin and cultured with low dose TP0903, cytokine production was favorably altered through the promotion of Th1 and reduction of Th2 cytokines. This was associated with a significant reduction of inhibitory receptors (FIG. 19A). Western blot of T cell lysates suggests low dose TP0903 results in inhibition of LCK. When effector T cells and regulatory T cells (Treg) were treated with TP0903 for 3 days, there was a preferential reduction of Treg (FIG. 19B).

The influence of TP0903 on CART19 cell phenotype and functions was investigated. Here, 41BB costimulated, lentiviral-transduced CART cells were used. Similar to the findings on naïve T cells, TP0903 treatment led to polarization of CART cells into a Th1 phenotype when T cells were stimulated with the CD19⁺ mantle cell lymphoma (MCL) cell line JeKo or with leukemic B cells isolated from CLL patients (FIG. 19C). TP0903 treatment also significantly downregulated inhibitory receptors on activated CART cells, including a reduction of canonical cytokines known to be associated with the development of cytokine release syndrome (CRS) (FIG. 19C). The combination of CART19 cells and TP0903 yielded a synergistic antitumor activity against JeKo in vitro, at low E:T ratios (FIG. 19E). Western blot of T cell lysates revealed phosphorylation of LCK was remarkably reduced in the presence of TP0903, suggesting a mechanism for the observed Th1 polarization. More than 100 genes were differentially expressed in the transcriptome of activated CART cells treated with TP0903 as compared to non-treated cells. Among these genes, immune synapse related genes such as cell junction and cell migration related genes were significantly increased in activated CART cells treated with TP0903.

To investigate the effect of AXL RTK inhibition of CART cells with TP0903 in vivo, MCL xenografts were established through the injection of 1.0×10⁶ of JeKo into NSG mice. A week after the injection of JeKo, mice were treated with either vehicle alone, TP0903 (20 mg/kg/day) alone, 0.5×10⁶ of CART19 alone, or TP0903 (20 mg/kg/day)+0.5×10⁶ of CART19. Three weeks after the treatment, mice were rechallenged with 1.0×10⁶ of JeKo. Mice treated with CART19 and TP0903 rejected the JeKo tumor challenge while mice previously treated with CART19 alone redeveloped JeKo, suggesting that AXL inhibition enhanced CART cell persistence (FIG. 19F).

The preclinical findings were validated in a correlative analyses of Phase I clinical trial of TP0903 for patients with solid tumors (NCT02729298). Blood T cells from 3 patients were isolated and analyzed before and a week after treatment with TP0903. Similar to the findings described above, there was a significant reduction in Tregs, reduction of inhibitory receptors and polarization to a Th1 phenotype. These findings are further investigated in a Phase I clinical trial of TP0903 in relapsed/refractory CLL (NCT03572634).

These results demonstrated that an AXL inhibitor can polarize T cells into a Th1 phenotype, downregulate inhibitory receptors, reduce CRS associated cytokines, and synergize with CART cells in B cell malignancies.

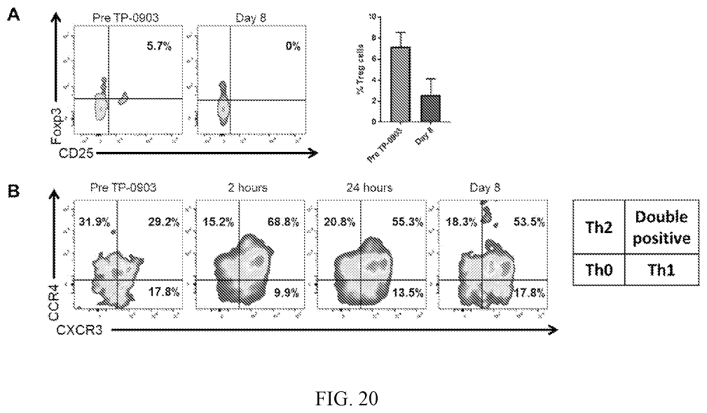

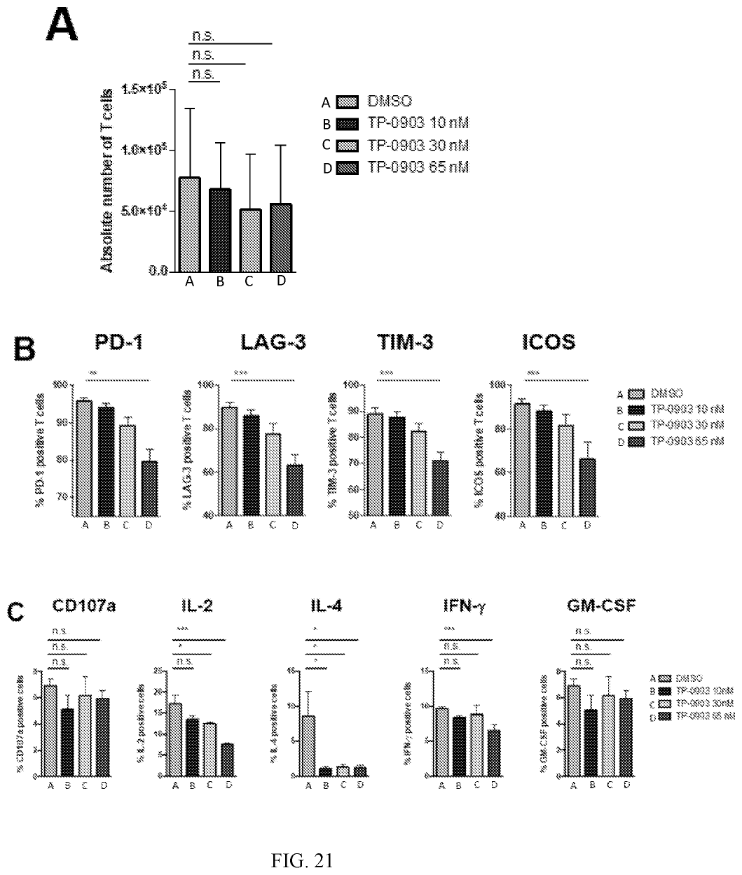

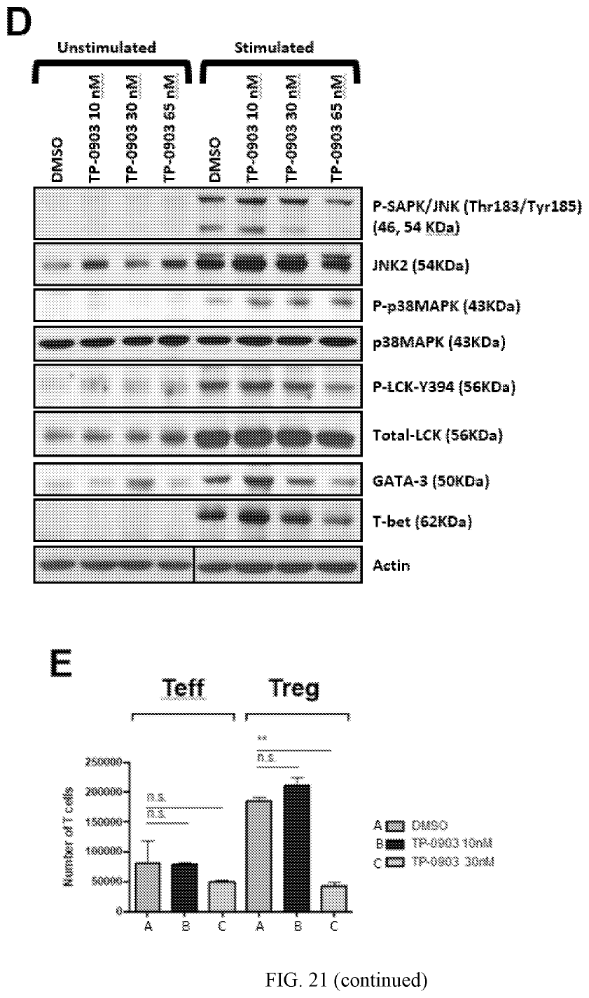

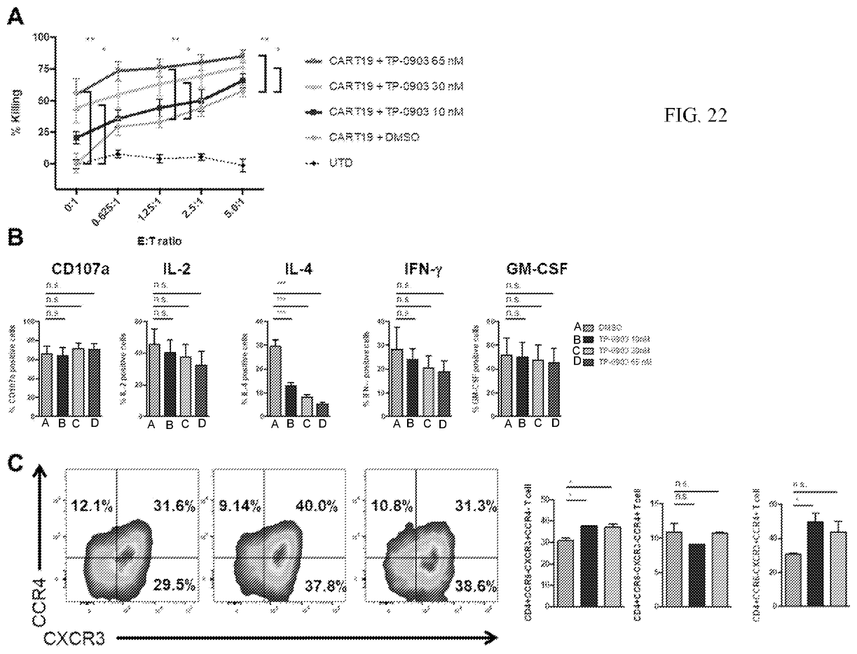

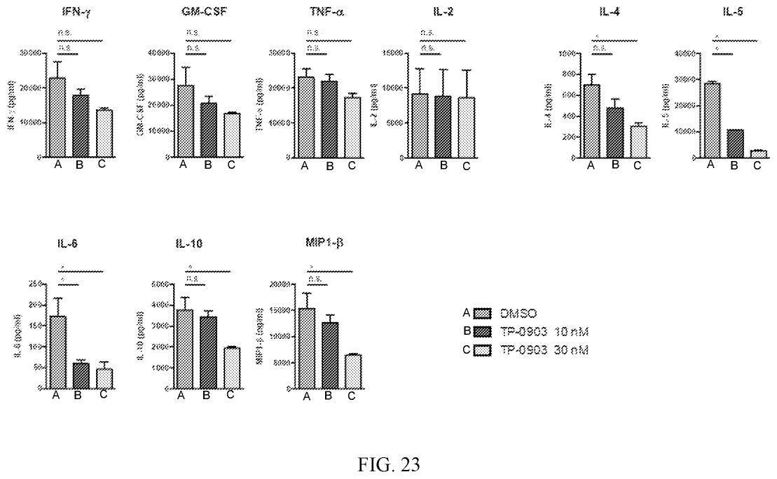

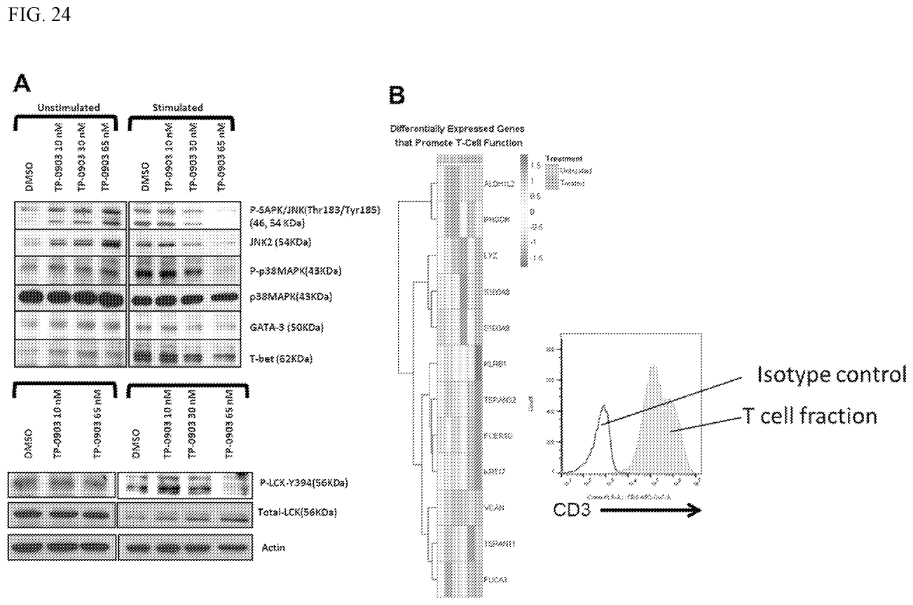

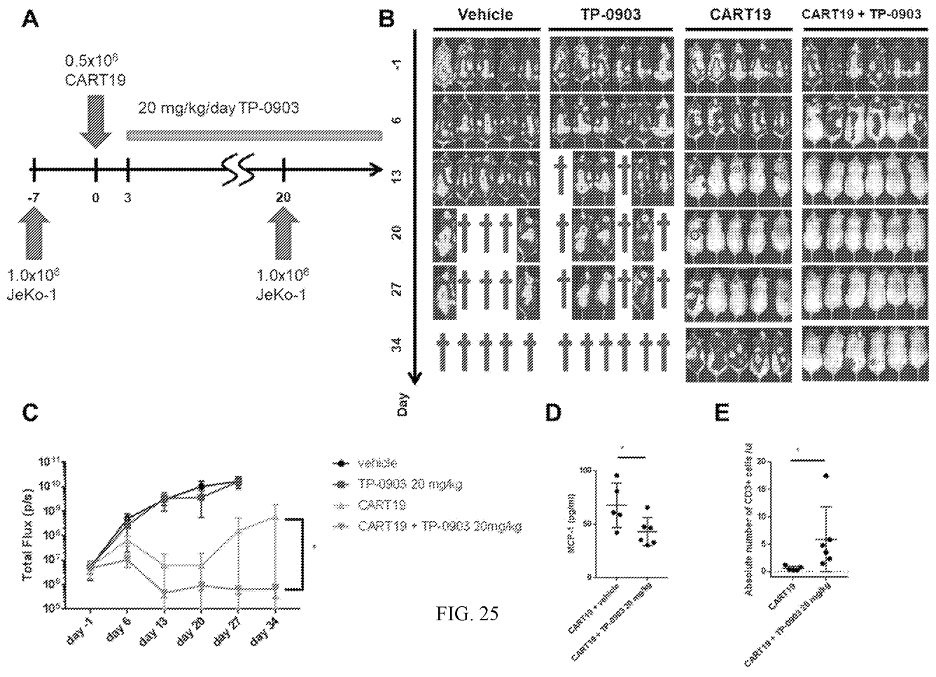

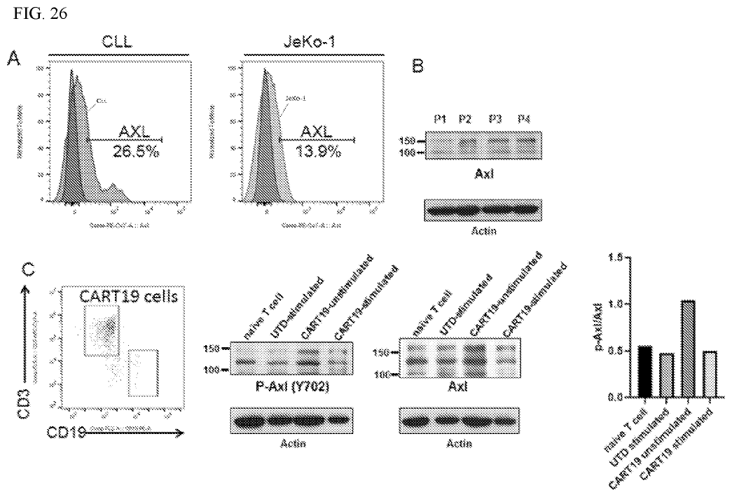

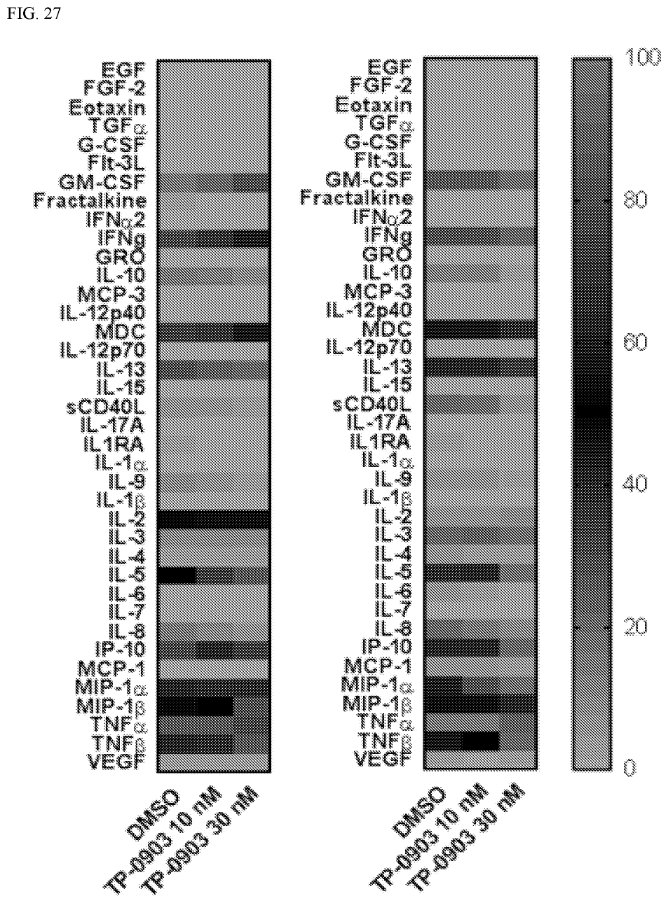

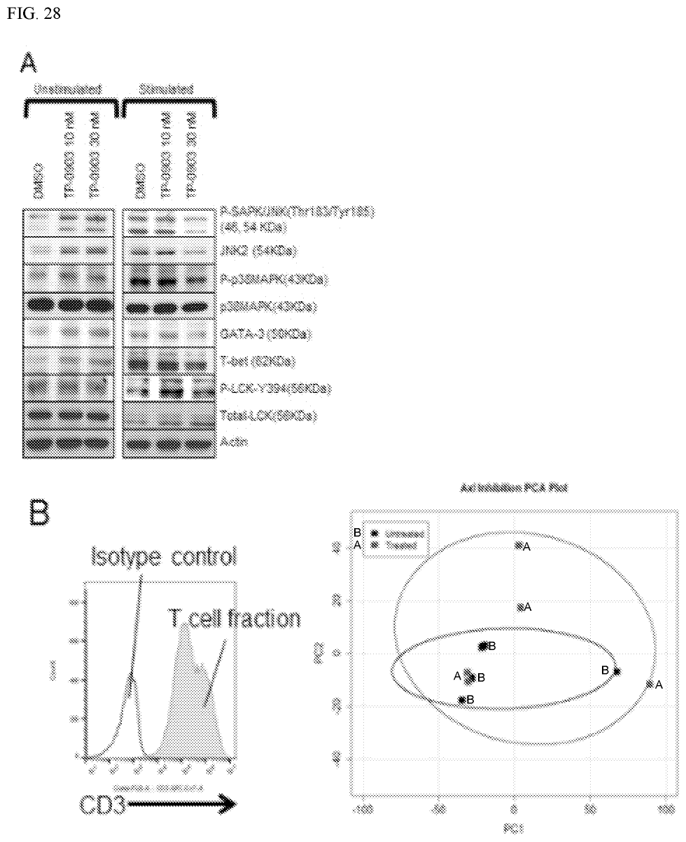

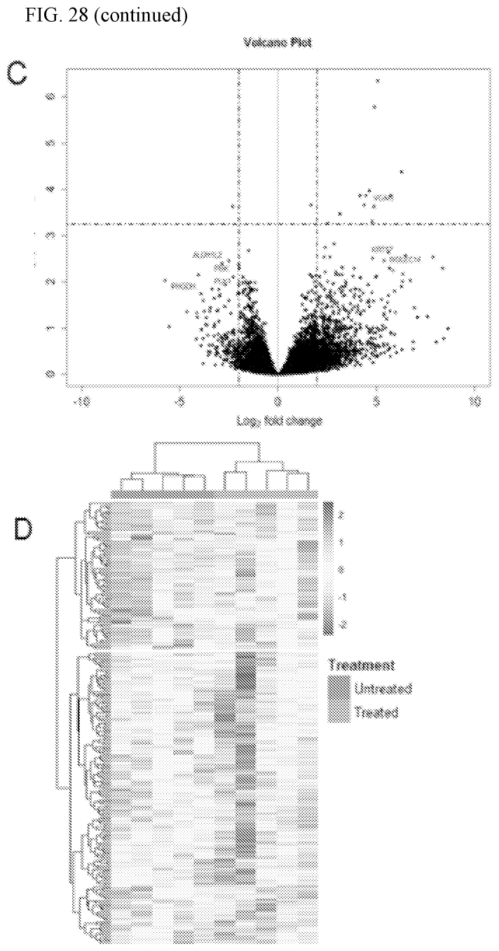

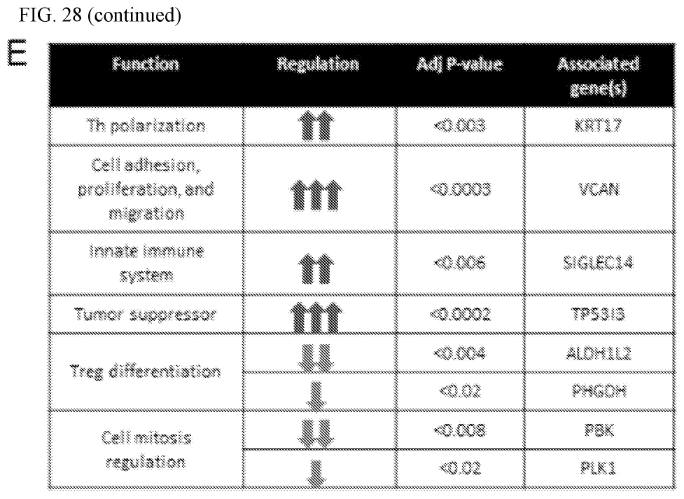

Additional results were provided in FIGS. 20-28.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating cancer, wherein said method comprises administering an AXL inhibitor to a mammal having cancer that received an adoptive T cell therapy to treat said cancer.

2. The method of claim 1, wherein said mammal is a human.

3. The method of claim 1, wherein said cancer is a lymphoma.

4. The method of claim 3, wherein said lymphoma is a diffuse large B cell lymphoma.

5. The method of claim 1, wherein said cancer is a leukemia.

6. The method of claim 5, wherein said leukemia is an acute lymphoblastic leukemia or a chronic lymphocytic leukemia.

7. The method of claim 1, wherein said AXL inhibitor is TP0903.

8-20. (canceled)

21. A method for treating cancer, wherein said method comprises administering T cells to a mammal having cancer, wherein said T cells were contacted with an AXL inhibitor.

22. The method of claim 21, wherein said mammal is a human.

23. The method of claim 21, wherein said cancer is a lymphoma.

24. The method of claim 23, wherein said lymphoma is a diffuse large B cell lymphoma.

25. The method of claim 21, wherein said cancer is a leukemia.

26. The method of claim 25, wherein said leukemia is an acute lymphoblastic leukemia or a chronic lymphocytic leukemia.

27. The method of claim 21, wherein said AXL inhibitor is TP0903.

28-40. (canceled)

41. An ex vivo method for expanding T cells for treating cancer, wherein said method comprises contacting T cells with an AXL inhibitor.

42. (canceled)

43. The method of claim 41, wherein said cancer is a lymphoma.

44. The method of claim 43, wherein said lymphoma is a diffuse large B cell lymphoma.

45. The method of claim 41, wherein said cancer is a leukemia.

46. The method of claim 45, wherein said leukemia is an acute lymphoblastic leukemia or a chronic lymphocytic leukemia.

47. The method of claim 41, wherein said AXL inhibitor is TP0903.

48-56. (canceled)
